(19) United States
(12) Patent Application Publication
(10) Pub. No.: US 2024/0241030 A1
(43) Pub. Date: Jul. 18, 2024

(54) INFORMATION PROCESSING SYSTEM, INFORMATION PROCESSING METHOD, NON-TRANSITORY COMPUTER-READABLE STORAGE MEDIUM AND SORTING SYSTEM

(71) Applicant: Sony Group Corporation, Tokyo (JP)

(72) Inventors: Kenji Yamane, Kanagawa (JP); Yasunobu Kato, Kanagawa (JP); Hirotaka Yoshida, Tokyo (JP)

(73) Assignee: Sony Group Corporation, Tokyo (JP)

(21) Appl. No.: 18/428,160

(22) Filed: Jan. 31, 2024

Related U.S. Application Data
(63) Continuation of application No. 17/613,009, filed on Nov. 19, 2021.

(30) Foreign Application Priority Data
May 28, 2019 (JP) 2019-099716

(51) Int. Cl. G01N 15/1429; G01N 15/10; G01N 15/14; G01N 15/149; G16B 40/10; G16B 40/20

(52) U.S. Cl. CPC G01N 15/1429; G01N 15/1459; G16B 40/10; G16B 40/20; G01N 2015/1006; G01N 2015/1477; G01N 15/149

(57) ABSTRACT

Techniques for sorting biological particles are described. The techniques may include applying a data compression process to data indicating light emitted from biological particles and outputting, based on a result of the data compression process, one or more groups of the biological particles to sort into additional groups of the biological particles. The techniques may further include using at least some of the data corresponding to the one or more groups of the biological particles in training at least one statistical model, wherein an output of the at least one statistical model specifies an indication to sort one or more of the biological particles.

### CROSS REFERENCE TO RELATED APPLICATIONS

The present application claims the benefit under 35 U.S.C. § 120 as a continuation application of U.S. Application Ser. No. 17/613,009, filed on Nov. 19, 2021, which claims the benefit under 35 U.S.C. § 371 as a U.S. National Stage Entry of International Application No. PCT/JP2020/021017, filed in the Japanese Patent Office as a Receiving Office on May 27, 2020, which claims priority to Japanese Patent Application Number JP2019-099716, filed in the Japanese Patent Office on May 28, 2019, the entire contents of each of which is hereby incorporated by reference in its entirety.

### TECHNICAL FIELD

The present disclosure relates to a sorting apparatus, a sorting system and a program.

### BACKGROUND ART

In the field of medicine or biochemistry, using a flow cytometer in order to speedily measure properties of a large number of particles is common. A flow cytometer is an apparatus that measures properties of each particle by applying rays of light to particles, such as flowing cells or beads, and detecting fluorescence, etc., that is emitted by the particles.

Apparatuses that sort particles that emit specific fluorescence from a measurement sample by controlling destinations to which the particles move based on fluorescence information that is detected by a flow cytometer have been also developed. Such sorting apparatuses are referred to as cell sorters.

In recent years, enabling flow cytometers to analyze particles more in detail by increasing the number of fluorescent substances that can be measured at a time has been considered. Increasing the number of fluorescent substances however increases the number of dimensions of measurement data, thereby complicating analysis by flow cytometers.

Various methods for flow cytometers to analyze measurement data have been considered. For example, the following Patent Literature 1 discloses a technique to estimate information on the shape of a biological subject based on a peak position of a pulse waveform that is detected from the biological subject to which rays of light are applied.

### CITATION LIST

### Patent Literature

### [PTL 1]

JP 2017-58361 A

### SUMMARY OF INVENTION

### Technical Problem

On the other hand, sorting apparatuses, such as cell sorters, are required to measure and analyze flowing particles and perform a process of determining whether to sort the particles based on the result of measurement and analysis within a limited time during which the particles flow in the apparatus.

Accordingly, sorting apparatuses, such as cell sorters, have been required to more speedily and in real time determine whether particles are particles to be sorted.

### Solution to Problem

According to the present application, some embodiments are directed to an information processing system comprising: at least one hardware processor; and at least one non-transitory computer-readable storage medium storing processor-executable instructions that, when executed by the at least one hardware processor, cause the at least one hardware processor to perform a method. The method comprises applying a data compression process to data indicating light emitted from biological particles; outputting, based on a result of the data compression process, one or more groups of the biological particles to sort into additional groups of the biological particles; and using at least some of the data corresponding to the one or more groups of the biological particles in training at least one statistical model, wherein an output of the at least one statistical model specifies an indication to sort one or more of the biological particles.

According to the present application, some embodiments are directed to an information processing method comprising: applying a data compression process to data indicating light emitted from biological particles; outputting, based on a result of the data compression process, one or more groups of the biological particles to sort into additional groups of the biological particles; and using at least some of the data corresponding to the one or more groups of the biological particles in training at least one statistical model, wherein an output of the at least one statistical model specifies an indication to sort one or more of the biological particles.

According to the present application, some embodiments are directed to at least one non-transitory computer-readable storage medium storing processor-executable instructions that, when executed by at least one hardware processor, cause the at least one hardware processor to perform: applying a data compression process to data indicating light emitted from biological particles; outputting, based on a result of the data compression process, one or more groups of the biological particles to sort into additional groups of the biological particles; and using at least some of the data corresponding to the one or more groups of the biological particles in training at least one statistical model, wherein an output of the at least one statistical model specifies an indication to sort one or more of the biological particles.

According to the present application, some embodiments are directed to a sorting system comprising: at least one hardware processor; and at least one non-transitory computer-readable storage medium storing processor-executable instructions that, when executed by the at least one hardware processor, cause the at least one hardware processor to perform a method. The method comprises obtaining data indicating the light received by the photodetector array; using the data and at least one statistical model to generate an output specifying an indication to sort one or more of the biological particles, wherein the at least one statistical model was trained using training data corresponding to one or more groups of biological particles determined based on a compressed format of the training data; and controlling a sorting apparatus based, at least in part, on the output to sort at least some of the biological particles.

According to the present application, some embodiments are directed to an information processing method comprising: obtaining data indicating light emitted from biological particles and received by a photodetector array; using the data and at least one statistical model to generate an output specifying an indication to sort one or more of the biological particles, wherein the at least one statistical model was trained using training data corresponding to one or more groups of biological particles determined based on a compressed format of the training data; and controlling a sorting apparatus based, at least in part, on the output to sort at least some of the biological particles.

According to the present application, some embodiments are directed to at least one non-transitory computer-readable storage medium storing processor-executable instructions that, when executed by at least one hardware processor, cause the at least one hardware processor to perform: obtaining data indicating light emitted from biological particles and received by a photodetector array; using the data and at least one statistical model to generate an output specifying an indication to sort one or more of the biological particles, wherein the at least one statistical model was trained using training data corresponding to one or more groups of biological particles determined based on a compressed format of the training data; and controlling a sorting apparatus based, at least in part, on the output to sort at least some of the biological particles.

### BRIEF DESCRIPTION OF DRAWINGS

FIG. 1 is a block diagram illustrating an exemplary configuration of a sorting system according to an embodiment of the disclosure.

FIG. 2A is an explanatory view to explain a detection mechanism of a filter system of a measurement unit.

FIG. 2B is an explanatory view to explain a detection mechanism of a spectrum system of the measurement unit.

FIG. 3 is a block diagram illustrating an exemplary configuration of an information processing apparatus according to the embodiment.

FIG. 4 is a table illustrating exemplary information on fluorescence of biological particles that is acquired from the sorting apparatus.

FIG. 5A is an explanatory view representing a result of a clustering.

FIG. 5B is an explanatory view representing the result of the clustering.

FIG. 6 is an explanatory view representing a result of performing dimensional compression to two dimensions on information on the levels of expression of respective fluorescent substances of the biological particles using the t-SNE algorithm.

FIG. 7 is a table representing information that is used as training data in machine learning by a learning unit.

FIG. 8A is a flowchart to explain a flow of operations to construct a learning model that are performed by the sorting system according to the embodiment.

FIG. 8B is a flowchart to explain a flow of operations to sort biological particles that are performed by The sorting system according to the embodiment.

FIG. 9A is an explanatory view illustrating an exemplary image that is represented to a user by a sorting system according to First Modification.

FIG. 9B is an explanatory view illustrating an exemplary image that is represented to the user by a sorting system according to First Modification.

FIG. 10 is a block diagram illustrating an exemplary configuration of a sorting system according to Second Modification.

FIG. 11 is a block diagram illustrating an exemplary configuration of an information processing apparatus and an information processing server according to Second Modification.

FIG. 12 is a block diagram illustrating an exemplary hardware configuration of an information processing apparatus according to an embodiment of the disclosure.

### DESCRIPTION OF EMBODIMENTS

Preferable embodiments of the disclosure are described in detail below with reference to the accompanying drawings. Note that redundant description of components having substantially the same functional configuration is omitted by assigning the same sign to the components herein and in the drawings.

Description will be given in the following order.
1. Configuration of sorting system
2. Configuration of information processing apparatus
3. Operations of sorting system
4. Modification of sorting system
5. Exemplary hardware configuration

### <1. Configuration of Sorting System>

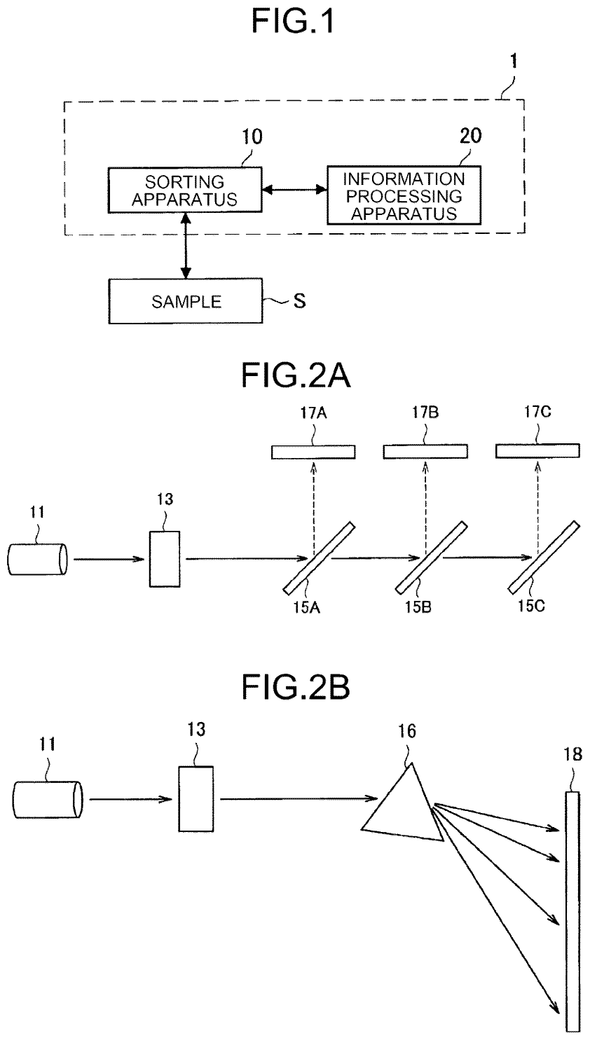

First of all, with reference to FIG. 1, a configuration of a sorting system 1 according to an embodiment of the disclosure will be described. FIG. 1 is a block diagram illustrating an exemplary configuration of the sorting system 1 according to the embodiment.

As illustrated in FIG. 1, the sorting system 1 according to the embodiment includes a sorting apparatus 10 that acquires measurement data from a sample S and that sorts particles to be sorted based on a determination made by an information processing apparatus 20; and the information processing apparatus 20 that analyzes the measurement data that is acquired by the sorting apparatus 10 and determines whether the particles are particles to be sorted. The sorting system 1 according to the embodiment is usable as, for example, a so-called cell sorter.

The sample S is, for example, biological particles, such as cells, microorganisms or organism-related particles, and contains multiple groups of biological particles. By analyzing the measurement data on the sample S, the sorting apparatus 10 is able to classify the biological particles into multiple groups of internal cohesion and external isolation and sort a specific classified group. The sample S may be, for example, cells like animal cells (for example, blood cells) or plant cells; microorganisms, such as bacteria like Escherichia coli, viruses like tobacco mosaic virus, or fungi like yeast; biological particles forming cells, such as chromosome, liposome, mitochondria, or various types of organelle; or biological fine particles, such as biological polymer like nucleic acid, protein, lipid, glycan or a compound thereof.

The sample S is labeled (colored) with at least one fluorescent dye. Labelling the sample S with a fluorescent dye can be performed by a known method. For example, when the sample S is cells, mixing fluorescent labeling antibodies that selectively combine with antigens existing on the surfaces of cells with the cells to be measured to combine the fluorescent labeling antibodies with the antigens on the surfaces of cells makes it possible to label the cells to be measured with the fluorescent dye.

The fluorescent labeling antibodies are antibodies with which fluorescent dyes are combined as labels. Specifically, the fluorescent labeling antibodies may be obtained by combining fluorescent dyes with which avidin is combined with antibodies labeled with biotin by avidin-biotin reaction. Alternatively, the fluorescent labeling antibodies may be obtained by directly combining fluorescent dyes with antibodies. Any of polyclonal antibodies and monoclonal antibodies may be used as the antibodies. The fluorescent dyes for labelling cells are also not particularly limited and it is possible to use at least one of known dye that is used to stain cells, etc.

The sorting apparatus 10 includes a measurement unit and a sorting unit. The sorting apparatus 10 may be the sorting apparatus 10 of a so-called flow cell type or may be a sorting apparatus of a microchannel chip type.

The measurement unit measures fluorescence that is emitted from the sample S because of application of rays of light, such as laser light, to the sample S. Specifically, the measurement unit causes laminar flow of a sheath fluid into which the sample S is dispersed, thereby aligning the sample S in one direction. The measurement unit applies laser light with a wavelength enabling the fluorescent dyes with which the sample S is labeled to the aligned sample S and performs photoelectric conversion on the fluorescence that is generated from the sample S to which the laser light is applied using a known photoelectric conversion device, such as a CCD (Charge Coupled Device), a CMOS (Complementary Metal Oxide Semiconductor), a photodiode, or a PMT (Photo Multiplier Tube). In this manner, the measurement unit is able to acquire fluorescence from the sample S.

The mechanism of the measurement unit to detect fluorescence from the sample S may be of any one of a filter system and a spectrum system. The mechanism to detect fluorescence from the sample S will be described with reference to FIGS. 2A and 2B. FIG. 2A is an explanatory view to explain a detection mechanism of the filter system and FIG. 2B is an explanatory view to explain a detection mechanism of the spectrum system.

As illustrated in FIG. 2A, using dichroic mirrors 15A, 15B and 15c, the detection mechanism of the filter system divides fluorescence obtained by applying rays of light from a light source 11 to the sample S flowing in a flow path 13. Accordingly, the detection mechanism of the filter system is able to acquire the intensity of fluorescence of each given wavelength band using photodetectors 17A, 17B and 17C.

Specifically, the dichroic mirrors 15A, 15B and 15C are mirrors that reflect light of given wavelength bands and transmit light of other wavelength bands. Thus, arranging the dichroic mirrors 15A, 15B and 15C that reflect light of different wavelength bands on an optical path of fluorescence from the sample S enables the measurement unit to separate the fluorescence according to the wavelength bands. For example, arranging the dichroic mirror 15A that reflects light of the wavelength band of red, the dichroic mirror 15B that reflects light of the wavelength band of green, and the dichroic mirror 15C that reflects light of the wavelength band of blue sequentially from the side on which the fluorescence from the sample S is incident enables the measurement unit to separate the fluorescence from the sample S according to the wavelength bands.

As illustrated in FIG. 2B, using a prism 16, the detection mechanism of the spectral system divides the fluorescence that is obtained by applying rays of light from the light source 11 to the sample S passing through the flow path 13. Accordingly, the detection mechanism of the spectrum system is able to acquire a continuous spectrum using an photodetector array 18.

Specifically, the prism 16 is an optical member that disperses light incident thereon. By dispersing the fluorescence from the sample S using the prism 16, the measurement unit enables detection of a continuous spectrum of fluorescence using the photodetector array 18 in which a plurality of photoelectric conversion elements are arranged in an array.

The sorting unit sorts part of the sample S to be sorted. Specifically, first of all, the sorting unit generates droplets of the sample S and charges the droplets of the sample S to be sorted. The sorting unit then moves the generated droplets into an electric field that is generated by a deflection plate. The charged droplets are attracted to the side of the charged deflection plate and accordingly the direction of move of the droplets changes. This enables the sorting unit to separate the droplets of the sample S to be sorted and droplets of the sample S not to be sorted from each other and thus sort biological particles to be sorted. The sorting system of the sorting unit may be any one of the jet in air system and a cuvette flow cell system. The sample S may be sorted by being ejected to the outside of the flow cell or the microchannel chip or may be sorted in the microchannel chip. Whether to perform separation on the sample S may be determined by a logic circuit (for example, FPGA (field-programmable gate array) circuit) of the sorting apparatus 10 or may be determined according to an instruction from the information processing apparatus 20.

The information processing apparatus 20 analyzes the measurement data on the sample S that is acquired by the measurement unit and represents the analyzed data to the user. The user is able to specify a group of biological particles to be sorted by checking the data that is analyzed by the information processing apparatus 20.

The information processing apparatus 20 analyzes the properties of biological particles by calculating levels of expression of the fluorescent dyes in the biological particles from the measurement data on the sample S. The number of dimensions of measurement data however increases in association with a recent increase in the number of colors in flow cytometers and thus a combinatorial explosion occurs, which makes it difficult for users to know each group of biological particles from the levels of expression of fluorescent dyes. Thus, techniques supporting the user in knowing groups of biological particles by data compression, etc., have been considered. The data compression herein represents not so-called lossless compression allowing compression and decompression but lossy compression. In other words, the data compression is processing that partly loses original data by compression but facilitates data analysis by reducing information.

Such data compression however may make it difficult to reproduce the data before processing from the data after the processing. For this reason, it has been difficult to derive what fluorescence information the group of biological particles that is specified by the user has based on the data after the data compression.

The information processing apparatus 20 thus has difficulty in setting conditions on making a determination on the measurement data on the group of biological particles to be sorted that is specified by the user based on the data after the data compression.

The sorting apparatus 10 measures fluorescence of biological particles that flows through the apparatus in real time and, based on the result of determination made by the information processing apparatus 20, sorts the biological particles whose fluorescence has been measured. For this reason, the information processing apparatus 20 is required to analyze the measurement data from the biological particles, then determine whether the biological particles are to be sorted, and output the result of determination to the sorting apparatus 10 within a limited time.

The amount of calculation for calculating levels of expression of fluorescent dyes in the biological particles however has become enormous in association with the recent increase in the number of colors in flow cytometers. Accordingly, the time required by the information processing apparatus 20 to calculate a level of expression of the fluorescent dye in biological particles from the measurement data on the sample S is also enormous. Additionally, the calculation time for the data compression described above is also enormous. Thus, it is not realistic that the information processing apparatus 20 executes the above-described analysis on each of the biological particles in real time while the sample S is flowing through the sorting apparatus 10 and calculates data after the data compression.

For this reason, a sorting system capable of analyzing what fluorescent information biological particles to be sorted that are specified by the user based on the data after the compression have and speedily determining whether the biological particles of the measurement data are to be sorted has been required.

In view of the above-described circumstances, the inventors have reached the technique according to the disclosure. The technique according to the disclosure enables a sorting system that sorts biological particles based on fluorescence to, by performing machine learning using information on biological particles to be sorted before data compression, determine whether the biological particles are to be sorted from fluorescence information.

According to the technique according to the disclosure, it is possible to speedily determine whether biological particles are to be sorted from fluorescent information on measured biological particles without performing complicated calculation. Accordingly, according to the technique according to the disclosure, it is possible to speedily determine whether biological particles are to be sorted not depending on the number of fluorescent substances with which biological particles are labeled and the method of analyzing measurement data.

### <2. Configuration of Information Processing Apparatus>

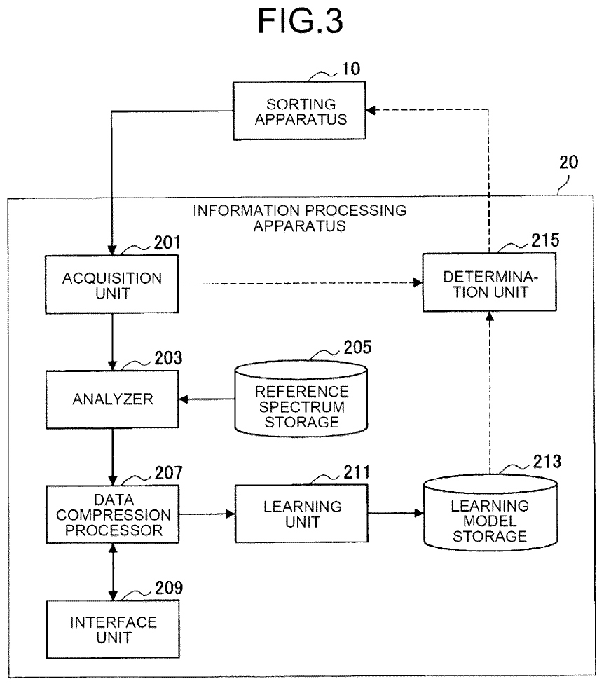

With reference to FIG. 3, a more specific configuration of the information processing apparatus 20 that the sorting system 1 according to the embodiment incudes will be described. FIG. 3 is a block diagram illustrating an exemplary configuration of the information processing apparatus 20 according to the embodiment.

As illustrated in FIG. 3, the information processing apparatus 2 includes an acquisition unit 201, an analyzer 203, a reference spectrum storage 205, a data compression processor 207, an interface unit 209, a learning unit 211, a learning model storage 213, and a determination unit 215.

The acquisition unit 201 acquires information on fluorescence of biological particles from the sorting apparatus 10. Specifically, the sorting apparatus 10 detects light of the biological particles using the detection mechanism of the spectrum system and the acquisition unit 201 acquires information on the spectrum of light of the biological particles. The light of the biological particles may be any one of scattering light and fluorescence from the biological particles to which laser light is applied or may be both of scattering light and fluorescence. The acquisition unit 201 may, for example, acquire the information on the light of the biological particles from the sorting apparatus 10 via a network, or the like, or may acquire information on the light of biological particles from the sorting apparatus 10 via a wired or wireless LAN (Local Area Network) or a wired cable.

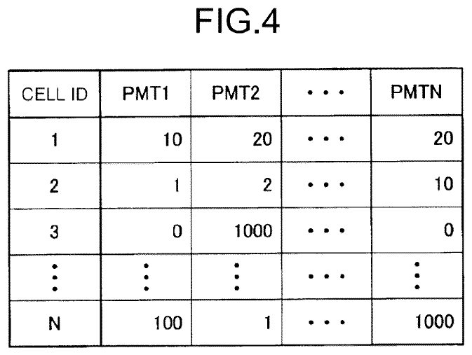

For example, the information on the light of the biological particles that is acquired by the acquisition unit 201 may be information like that represented in FIG. 4. FIG. 4 is a table representing exemplary information on the light of the biological particles that is acquired from the sorting apparatus 10.

As illustrated in FIG. 4, the information on the light of the biological particles may represent, for each identification number of cell (that is, biological particle), gains that are detected by respective N photo multiplier tubes (PMT) that are arranged in the photodetector array as “PMT1” to “PMTN”. The N photo multiplier tubes are arranged in line in an array in a direction in which light is dispersed by the prism. For this reason, sequentially arranging the gains of the N photo multiplier tubes as a histogram enables acquisition of a spectrum of light of the cell. FIG. 4 represents the results of measuring the gains of the N photo multiplier tubes respectively for the N cells.

The analyzer 203 derives information on properties of the biological particles measured by the sorting apparatus 10 by analyzing the information on the light of the biological particles. Specifically, by separating sets of fluorescence contained in the fluorescent spectrum measured by the sorting apparatus 10, the analyzer 203 derives the levels of expression of the fluorescent substances corresponding to the respective sets of fluorescence in the biological particles.

The biological particles to be measured are labelled with a plurality of fluorescent substances that emit fluorescence of wavelength distributions overlapping with each other. For this reason, by weighting the wavelength distribution of fluorescence that is emitted from each fluorescent substance and fitting the weighted wavelength distribution to the fluorescent spectrum that is measured by the sorting apparatus 10, it is possible to derive a level of expression of each of the fluorescent substance.

More specifically, first of all, the analyzer 203 acquires reference spectra respectively representing wavelength distributions of fluorescence that is emitted by the fluorescent substances with which the biological particles are labeled from the reference spectrum storage 205. The analyzer 203 then superimposes the reference spectra of the respective fluorescent substances and fits the superimposed fluorescent spectra to the fluorescent spectrum measured by the sorting apparatus 10 by the weighting least squares method, thereby being able to estimate the level of expression of each of the fluorescent substances.

The reference spectrum storage 205 stores each of the reference spectra representing the wavelength distributions of fluorescence that are emitted by the fluorescent substances that can label biological particles. Any one of the information processing apparatus 20 and the sorting apparatus 10 may include the reference spectrum storage 205 or another information processing apparatus or information processing server capable of communication via a network may include the reference spectrum storage 205.

The data compression processor 207 performs data compression on optical information on biological particles that is analyzed by the analyzer 203.

The data compression includes both non-linear processing and linear processing. For example, non-linear processing may include dimensional compression, clustering and grouping. For example, linear processing may include processing to generate fluorescent information on each fluorescent dye from spectral information on light of biological particles by performing separation of fluorescent.

For non-linear processing, an algorism of any of supervised or unsupervised machine learning or semi-supervised machine learning may be used. Note that it is desirable that the machine learning algorithm used for non-linear processing be different from a machine learning algorithm that is used by the learning unit 211 to be described below.

Specifically, the data compression processor 207 may perform clustering on information on the level of expression of each of the fluorescent substances of the biological particles. Clustering enables the data compression processor 207 to classify the biological particles into multiple groups of external isolation and internal cohesion.

An algorithm for clustering is not particularly limited, and a known clustering algorithm is usable. For example, the data compression processor 207 may perform clustering using an algorithm that allows specifying the number of clusters, such as k-means, or may perform clustering using an algorithm that automatically determines the number of clusters, such as flowsom.

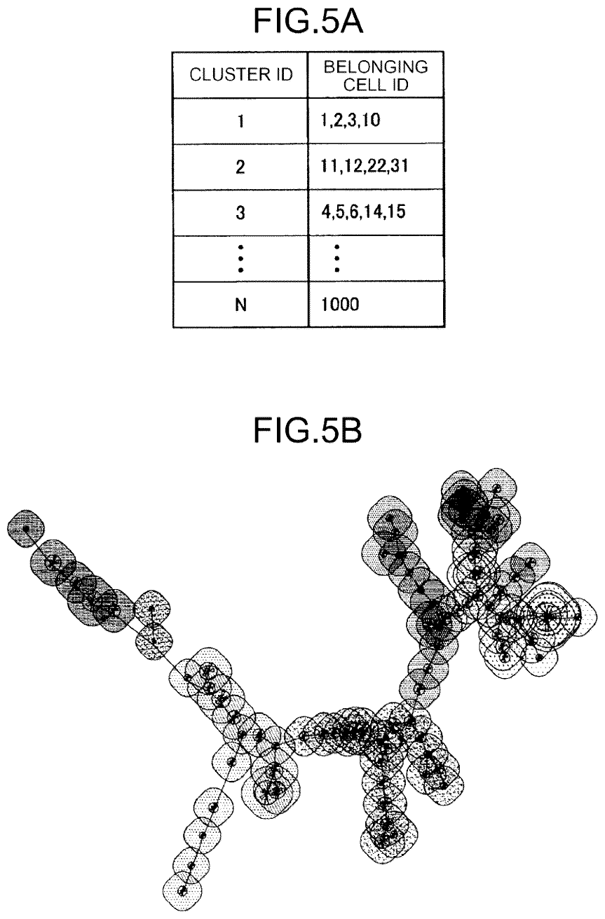

The result of clustering performed by the data compression processor 207 may be represented to the user in a form like that represented in FIG. 5A and FIG. 5B. FIG. 5A and FIG. 5B are an explanatory views representing a result of clustering.

For example, as illustrated in FIG. 5A, the result of clustering performed by the data compression processor 207 may be represented in a form of table to the user.

In FIG. 5A, a group of 1,000 cells (that is, biological particles) are divided into N clusters and affiliation of cells with each cluster is represented by identification numbers that are assigned to the clusters and cells, respectively. Specifically, in FIG. 5A, the cells of the identification numbers “1”, “2”, “3” and “10” belong to the cluster of the identification number “1”, the cells of the identification numbers “11”, “12”, “22” and “31” belong to the cluster of the identification number “2”, the cells of the identification numbers “4” to “6”, “14”, and “15” belong to the cluster of the identification number “3”, and the cell of the identification number “1000” belongs to the cluster of the identification number “N”. Representation to the user using such a form of table enables simple representation of affiliation of cells with each cluster.

For example, as illustrated in FIG. 5B, the result of clustering performed by the data compression processor 207 may be represented in a form of minimum spanning tree to the user.

In FIG. 5B, radar charts that are differently colored with a plurality of colors (in FIG. 5B, colors are distinguished according to the type of hatching) are arrayed like a tree in which the radar charts are connected. Each radar chart represents each cell (that is, biological particle). Specifically, the distribution and size of each radar chart represents a vector corresponding to the level of expression of each fluorescent substance of the cell. The areas differently colored in the respective colors represent clusters to which each cell belongs. It is represented that, for example, cells represented by radar charts that are colored in the same color (that is, the same type of hatching) belong to the same cluster.

In FIG. 5B, the distance between radar charts correspond to similarity between the cells represented by the radar charts. In other words, FIG. 5B represents that cells represented by radar charts close to each other are similar to each other and cells represented by radar charts apart from each other are not similar to each other. According to representation in such a form of minimum spanning tree enables representation of relationships in similarity between cells in addition to affiliation of cells with clusters.

Alternatively, the data compression processor 207 may perform dimensional compression on information on the level of expression of each fluorescent substance of the biological particles. The dimensional compression enables the data compression processor 207 to, by compressing dimensions of high-dimensional data containing the levels of expression of a plurality of fluorescent substances, visualize each relationship of high-dimensional data on a low-dimensional map such that the relationship is easily understandable. Accordingly, by checking the low-dimensional information after the dimensional compression, the user is able to classify biological particles into multiple groups more easily than with high-dimensional data before dimensional compression. The data compression processor 207 preferably performs dimensional compression to reduce the number of dimensions by at least one and, for example, by compressing the dimensions of information on the levels of expression of the respective fluorescent substances of the biological particles into three dimensions or less, the data compression processor 207 is able to visualize the relationships in high-dimensional data more clearly.

An algorithm for dimensional compression is not particularly limited, and a known dimensional compression algorithm is usable. For example, the data compression processor 207 may perform dimensional compression using an algorithm, such as PCA, t-SNE or Umap.

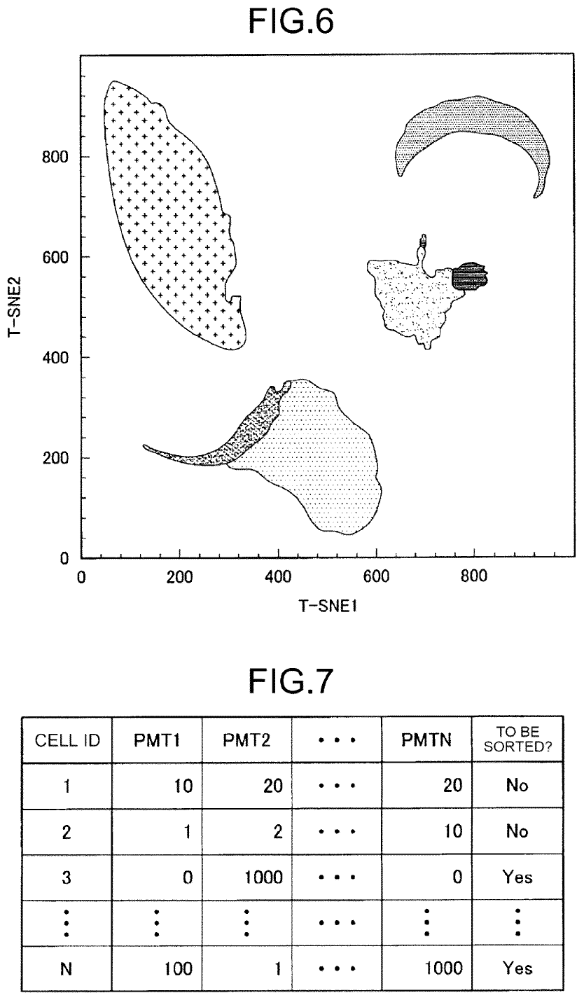

The result of dimensional compression performed by the data compression processor 207 may be represented in a form like that illustrated in FIG. 6. FIG. 6 is an explanatory view representing the result of performing dimensional compression to two dimensions on the information on the levels of expression of the respective fluorescent substances of the biological particles using the t-SNE algorithm.

For example, in FIG. 6, Euclid distances of high-dimensional data that are the levels of expression of the respective fluorescent substances of the cells are converted into rates using a rate distribution of a t-distribution of a student and are mapped onto two-dimensional coordinates. This allows the user to compare similarities in level of expression between the fluorescent substances of the cells in a more simplified manner without comparing the levels of expression of the respective fluorescent substances. For example, FIG. 6 represents cells that belong to the same group in different colors (colors are distinguished according to the type of hatching in FIG. 6). With reference to FIG. 6, it is represented that the dimensional compression appropriately groups cells that belong to the same group by internal cohesion and external isolation.

The interface unit 209 includes an output device and an input device and inputs and outputs information to and from the user. Specifically, the interface unit 209 may represent information after non-linear processing performed by the data compression processor 207 using a display device, such as a CRT (Cathode Ray Tube) display device, a liquid crystal display device or an OLED (Organic Light Emitting Diode) display device, or the like. The interface unit 209 may receive an input to specify biological particles to be sorted from the user using an input device, such as a touch panel, a keyboard, a mouse, a button, a microphone, a switch or a lever.

The user is able to more easily specify a group of biological particles to be sorted by checking the information after data compression that is output from the interface unit 209. For example, by checking the information after clustering, the user is able to specify a cluster of biological particles to be sorted. Furthermore, the user is able to specify a range of a group of biological particles to be sorted.

The learning unit 211 performs machine learning using information before data compression on the biological particles to be sorted, thereby constructing a learning model to determine whether biological particles are to be sorted using information on light that is emitted from the biological particles.

The constructed learning model may be, for example, stored in the learning model storage 213 that the information processing apparatus 20 includes. This enables the sorting apparatus 10 to sort biological particles to be sorted according to separation control from the information processing apparatus 20. Alternatively, the constructed learning model may be installed in a logic circuit, such as a FPGA circuit, that is arranged in the sorting apparatus 10. For example, the determination unit 215 may be arranged in the sorting apparatus 10 and a logic to execute the learning model that is designed and constructed based on the type of the determination unit 215 may be installed in the FPGA circuit that is arranged in the sorting apparatus 10. The learning unit 211 may design the logic to execute the constructed learning model.

The sorting system 1 according to the embodiment sorts the group of biological particles that is specified by the user as one to be sorted. The data compression that is performed by the data compression processor 207 however is lossy compression and thus it is difficult to derive information before processing performed by the data compression processor 207 from information after the processing. For this reason, when the user specifies biological particles to be sorted based on the information after data compression, it is difficult to derive what light the biological particles to be sorted emit. Thus, the information processing apparatus 20 has difficulty in determining conditions on determining whether biological particles are biological particles to be sorted.

By performing machine learning using the information before data compression on the group of biological particles that is specified by the user as one to be sorted, the sorting system 1 constructs a learning model to determine whether the biological particles are biological particles to be sorted. Specifically, the learning unit 211 is able to construct a learning model to determine whether biological particles are biological particles to be sorted by performing machine learning using, as training, information on the spectrum of light of the biological particles that are specified as biological particles to be sorted.

The learning unit 211 may construct a learning model to determine whether biological particles are biological particles to be sorted by performing machine learning using information on the level of expression of each fluorescent substance of the biological particles that are specified as biological particles to be sorted.

Note that deriving the level of expression of each fluorescent substance by the analyzer 203 requires an enormous volume and time of calculation in association with labelling biological particles with a large number of colors. Thus, analysis by the analyzer 203 from the information on fluorescent spectra of biological particles to information on the level of expression of each fluorescent substance also takes an enormous time. When the sorting apparatus 10 actually sorts biological particles, it is important to determine whether the biological particles are to be sorted within a limited time. Thus, constructing a learning model using the fluorescent spectrum of the biological particles that is measured by the sorting apparatus 10 better enables construction of a learning model to speedily determine whether biological particles are to be sorted.

The algorithm for machine learning that is performed by the learning unit 211 is supervised learning using information, as training, information on the fluorescent spectrum of the biological particles that are specified as biological particles to be sorted. For example, the learning unit 211 may construct a learning model using a learning algorithm, such as random forests, support vector machine, or deep learning. In some embodiments, the learning unit 211 may generate one or more statistical models using one or more suitable machine learning algorithms, including one or more classifiers. Examples of classifiers that a statistical model may include are a random forest classifier and a support vector machine classifier.

The sorting system 1 according to the embodiment uses various types of information that are not standardized as training and therefore a machine learning algorithm of random forests that does not require standardization can be preferably used. The random forests learning algorithm enables allows learning models to be easily executable by hardware and therefore the random forests learning algorithm can be preferably used for the sorting system 1 according to the embodiment in which it is important to speedily determine whether biological particles are to be sorted.

The information that is used for machine learning by the learning unit 211 may be, for example, information like that represented in FIG. 7. FIG. 7 is a table representing information that is used as training data in machine learning by the learning unit 211.

As illustrated in FIG. 7, the information that is used for machine learning may be information representing, for each identification number of cell (biological particle), gains that are detected by the N respective photo multiplier tubes (PMTs) that are arranged in the photodetector array as “PMT1” to “PMTN” and whether the cell is to be sorted by “Yes” (to be sorted) or “No” (not to be sorted) in the “to be sorted?” row. Using such information enables the learning unit 211 to construct the learning model having learned the characteristics of the gains of the respective photo multiplier tubes for the cells to be sorted.

The learning unit 211 may determine whether a learning model that sufficiently enables separation determination has been constructed and notify the user of the determination. For example, the learning unit 211 may, when the number of sets of information on biological particles having learned or the ratio of the number of sets of information to the whole exceeds a threshold, notify the user that a learning model that sufficiently enables separation determination has been constructed.

Alternatively, when the rate of correct answers of the learning model exceeds a threshold, the learning unit 211 may notify the user that a learning model that sufficiently enables separation determination has been constructed. The rate of correct answers of the learning model can be determined by, for example, N-fold-cross validation. Specifically, the rate of correct answers of the constructed learning model can be determined by, after dividing the whole information to be used as training into N sections and performing learning using information contained in the divided N−1 sections, making a determination on information contained in the remaining one divided section.

The learning model storage 213 stores the learning model that is constructed by the learning unit 211. The learning model storage 213 may store the learning model that is made executable by hardware using a FPGA (Field-Programmable Gate Array) circuit. This enables more speedy determination on whether biological particles are to be sorted.

The determination unit 215 determines whether the biological particles that emit fluorescence measured by the sorting apparatus 10 are to be sorted, based on the learning model that is stored in the learning model storage 213. When it is determined that the biological particles are to be sorted, the determination unit 215 issues an instruction to sort the biological particles to the sorting apparatus 10.

The learning model storage 213 and the determination unit 215 may be arranged in the sorting apparatus 10.

When the sorting apparatus 10 is able to sort multiple groups of biological particles separately, the determination unit 215 may issues an instruction indicating, in addition to whether the biological particles are to be sorted, in which collecting unit the biological particles are collected. In such a case, the learning unit 211 performs machine learning using, as training data, information on a fluorescent spectrum of biological particles on which into which collection unit the biological particles are collected is further specified. This enables the determination unit 215 to output an instruction to sort the multiple groups of biological particles separately to the sorting apparatus 10.

The above-described configuration enables the sorting system 1 according to the embodiment to, based on the information before data compression, speedily sort the biological particles to be sorted that are specified based on the information after data compression.

On the contrary, by performing machine learning using the information before data compression on biological particles not to be sorted, the sorting system 1 according to the embodiment may determine biological particles not to be sorted based on the information before data compression. Even in such a case, by separating biological particles other than the determined biological particles, the sorting system 1 according to the embodiment is able to sort biological particles to be sorted speedily.

### <3. Operations of Sorting System>

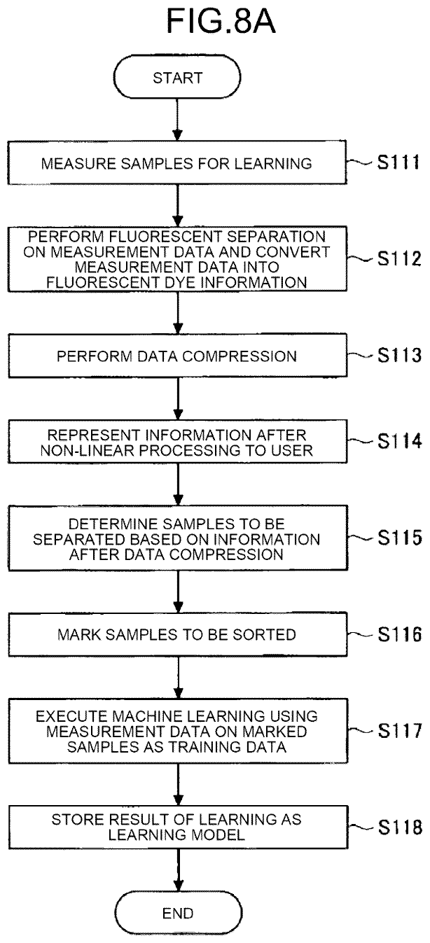

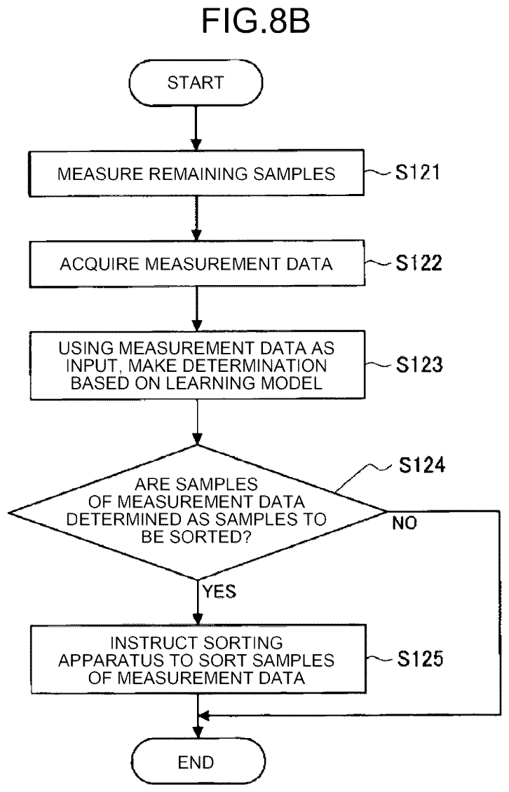

With reference to FIG. 8A and FIG. 8B, a flow of operations of the sorting system 1 according to the embodiment will be described. FIG. 8A is a flowchart to explain a now of operations to construct a learning model that are performed by the sorting system 1 according to the embodiment. FIG. 8B is a flowchart to explain a flow of operations to sort biological particles that are performed by the sorting system 1 according to the embodiment.

When the sorting system 1 according to the present embodiment constructs a learning model, as illustrated in FIG. 8A, first of all, the sorting apparatus 10 measures samples of biological particles for learning (S111). The information processing apparatus 20 acquires measurement data on the samples via the acquisition unit 201 and, using the analyzer 203, performs fluorescent separation on the measurement data, thereby deriving information on the level of expression of each fluorescent substance (that is, fluorescent dye information)(S112). Using the data compression processor 207, the information processing apparatus 20 then performs data compression on the fluorescent dye information (S113). Thereafter, the information processing apparatus 20 represents the information after data compression to a user via the interface unit 209 (S114).

The user refers to the represented information after data compression, thereby specifying a group of samples to be sorted (S115). Accordingly, using the learning unit 211, the information processing apparatus 20 marks, as samples to be sorted, the samples that are specified as samples to be sorted (S116). Using the learning unit 211, the information processing apparatus 20 then executes machine learning using the measurement data marked as samples to be sorted as training data (S117). After performing machine learning using a sufficient number of sets of training data, the information processing apparatus 20 stores a learning model that is constructed by machine learning in the learning model storage 213 (S118).

On the other hand, when the sorting system 1 according to the embodiment sorts biological particles, as illustrated in FIG. 8B, first of all, the sorting apparatus 10 measures samples of remaining biological particles for separation (S121). The information processing apparatus 20 subsequently acquires measurement data on the samples via the acquisition unit 201 (S122). Using the acquired measurement data as an input, the information processing apparatus 20 then makes a determination on whether the samples of the measurement data are to be sorted based on the learning model that is constructed by machine learning (S123).

Using the determination unit 215, the information processing apparatus 20 checks whether the samples of the measurement data are determined as samples to be sorted (S124) and, when it is determined that the samples of the measurement data are to be sorted (S124/Yes), outputs an instruction to sort the samples of the measurement data to the sorting apparatus 10 (S125). On the other hand, when it is determined that the samples of the measurement data are not to be sorted (S124/No), the instruction to sort the samples of the measurement data is not output and thus the sorting apparatus 10 does not sort the samples of the measurement data.

According to the flow of the operations above, the sorting system 1 according to the embodiment is able to speedily determine whether biological particles are to be sorted based on the learning model that is constructed by machine learning.

### <4. Modification of Sorting System>

### First Modification

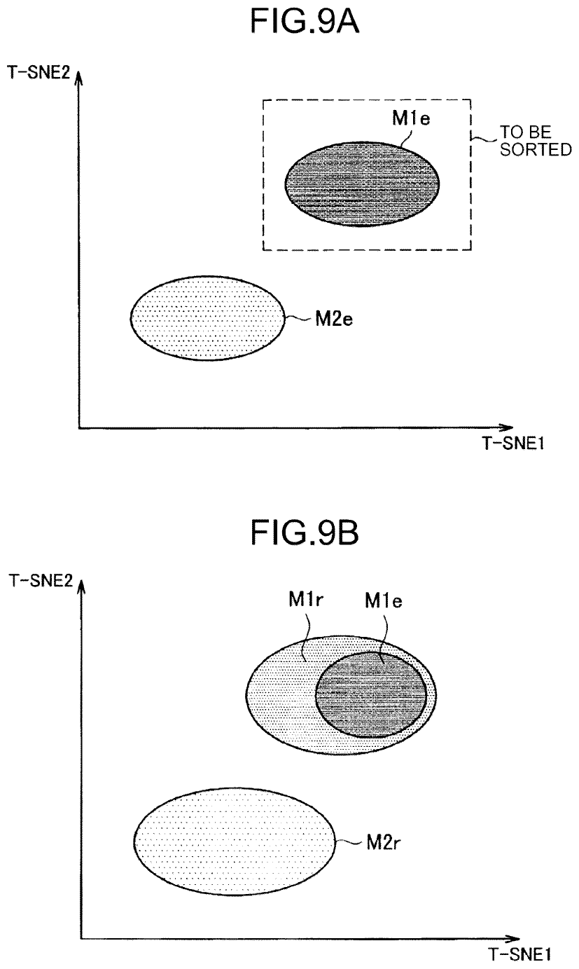

With reference to FIG. 9A and FIG. 9B, First Modification of the sorting system 1 according to the embodiment will be described. FIG. 9A and FIG. 9B are explanatory views illustrating an exemplary image that is represented to the user by the sorting system 1 according to First Modification.

The sorting system 1 according to First Modification stores, as information on biological parties, in addition to the measured gains of the photo multiplier tubes like those represented in FIG. 7 and the information on whether the biological particles are to be sorted, various types of information, such as identification numbers of clusters to which biological particles belong, parameters after dimensional compression, information on whether biological particles are used as training data for machine learning, information on whether biological particles are sorted actually, and the level of expression of each fluorescent substance after fluorescent separation, in association with one another.

For example, after separating biological particles, the user may check whether a group of biological particles that are sorted actually and a group of biological particles that are specified as biological particles to be sorted are similar to each other. This is because the number of biological particles of a population and the sampling timing among the samples differ between the measurement data on biological particles that are used for machine learning and measurement data on biological particles that are actually sorted and therefore the distribution of measurement data may differ.

Thus, the information processing apparatus 20 stores, for each biological particle, a cluster identification number in machine learning or a parameter after dimensional compression, and information on whether the biological particle is used for machine learning and on whether the biological particle is sorted actually in association with each other. This enables the information processing apparatus 20 to, after measuring all samples ends, perform the same processing on the distribution of biological particles used as biological particles to be sorted for machine learning and the distribution of biological particles that are sorted actually and then represent the distributions in a superimposed manner to the user.

With reference to FIG. 9A and FIG. 9B, more specific explanation will be given. For example, the distribution after dimensional compression on measurement data of samples for machine learning is sorted into groups M1e and M2e as in the graph represented in FIG. 9A and the group M1e is specified as a group to be sorted. The learning unit 211 thus performs machine learning using the measurement data on the group M1e as training data, thereby constructing a learning model. Thereafter, in the information processing apparatus 20, the determination unit 215 applies the learning model using the measurement data on the samples for separation as an input and outputs an instruction to sort the biological particles that are determined as particles to be sorted to the sorting apparatus 10.

According to First Modification, it is possible to, after separation of samples ends, represent the distribution after the same dimensional compression on all the measurement data to the user as illustrated in FIG. 9B. Accordingly, the user is able to check whether the distribution of the group M1e of biological particles used as training data for machine learning and the distribution of a group M1r of biological particles that are sorted actually overlap when the same processing is performed on the distributions. Furthermore, the user is able to check whether the distribution of the group M1r of biological particles that are sorted actually and the distribution of the group M2e of other biological particles separate from each other.

### Second Modification

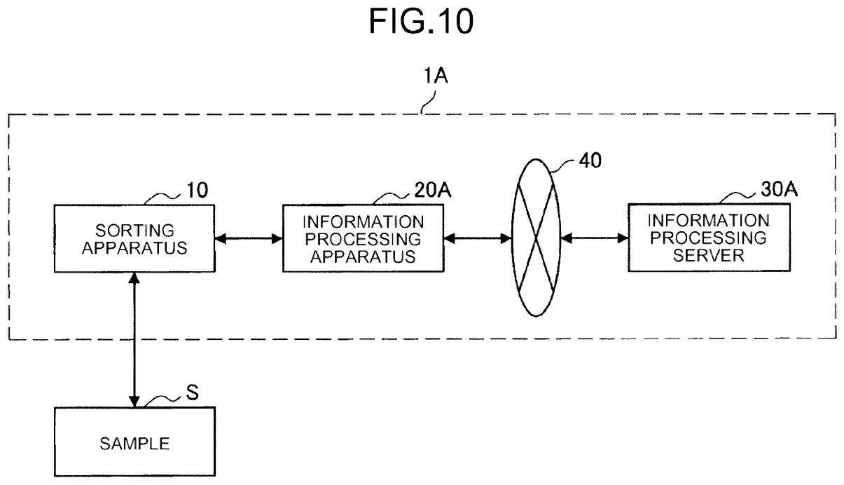

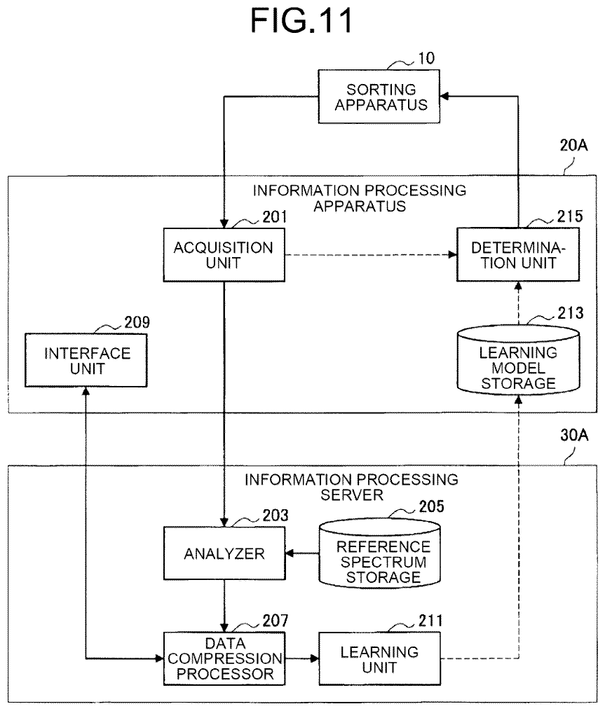

With reference to FIG. 10 and FIG. 11, Second Modification of the sorting system 1 according to the present embodiment will be described. FIG. 10 is a block diagram illustrating an exemplary configuration of a sorting system 1A according to Second Modification. FIG. 11 is a block diagram illustrating an exemplary configuration of an information processing apparatus 20A and an information processing server 30A according to Second Modification.

The sorting system 1A according to Second Modification is an example where the functions of the information processing apparatus 20 illustrated in FIG. 3 are separately imparted to a plurality of devices that are the information processing device and the information processing server.

Specifically, as illustrated in FIG. 10, the sorting system 1A according to Second Modification includes the sorting apparatus 10 that acquires measurement data from a sample S and that sorts particles to be sorted based on a determination made by the information processing apparatus 20A, the information processing apparatus 20A that determines whether particles are to be sorted, and the information processing server 30A that analyzes the measurement data that is acquired by the sorting apparatus 10. The information processing apparatus 20A and the information processing server 30A are connected with each other such that they can communicate with each other via a network 40, such as a public network like the Internet, a telephone network, or a satellite communication network, various types of LAN (Local Area Network) including Ethernet (Trademark) or WAN (Wide Area Network).

For example, as illustrated in FIG. 11, the information processing apparatus 20A may include the acquisition unit 201, the interface unit 209, the learning model storage 213, and the determination unit 215, and the information processing server 30A may include the analyzer 203, the reference spectral storage 205, the data compression processor 207, and the learning unit 211.

In the sorting system 1A according to Second Modification, it is possible to put devices with large computational capacity (for example, the analyzer 203, the data compression processor 207, and the learning unit 211) in charge of functions of larger computational loads. On the other hand, the information processing apparatus 20A that is directly connected to the sorting apparatus 10 may be put in charge of the functions of the determination unit 215 and the learning model storage 213 because delay due to the network 40, or the like, is desirably deviated for speedy determination and the computational load is not large.

If there is a purpose of speedy determination, the sorting apparatus 10 may include the determination unit 215 and the learning model storage 213. In such a case, in one of the information processing apparatus 20A and the information processing server 30A, a logic that realizes the learning model that is constructed by the learning unit 211 is designed based on the type of the determination unit 215. Thereafter, the designed logic is transmitted to the sorting apparatus 10 and thus is installed in a FPGA circuit of the sorting apparatus 10. This enables the sorting system 1A according to Second Modification to speedily determine biological articles to be sorted.

The configuration of the sorting system according to the embodiment of the disclosure is not limited to the configurations that are exemplified in FIG. 3 and FIG. 10. For example, the sorting system according to the embodiment may be formed of only the sorting apparatus 10. Specifically, the sorting apparatus 10 may further include the functions of the information processing apparatus 20. The sorting apparatus 10 may be given a learning model that is constructed by a computer that operates according to a program that is loaded into the computer and thus fulfill the functions of the information processing apparatus 20 and accordingly be able to sort biological particles to be sorted.

### <5. Exemplary Hardware Configuration>

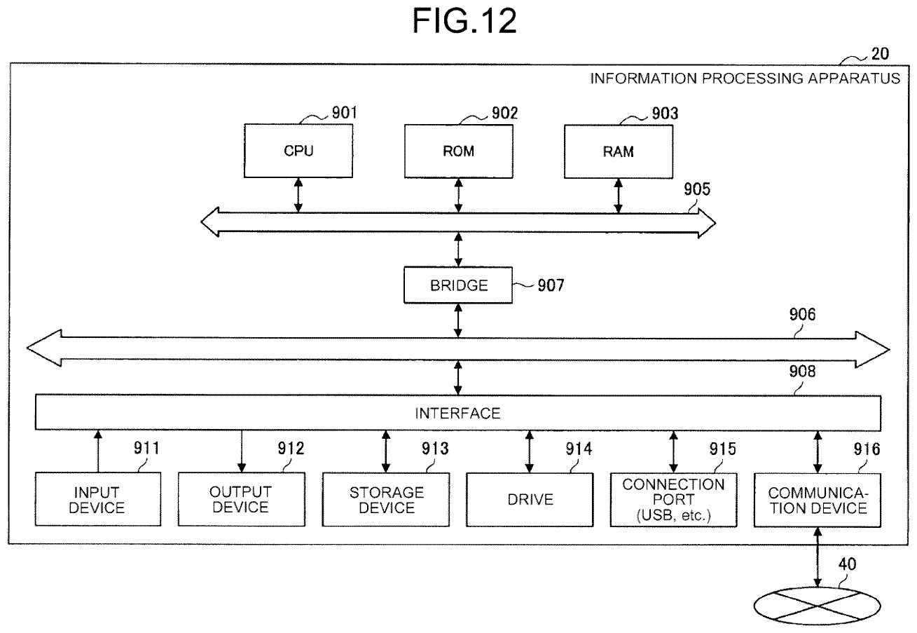

With reference to FIG. 12, an exemplary hardware configuration of the information processing apparatus 20, etc., according to the embodiment will be described. FIG. 12 is a block diagram illustrating an exemplary hardware configuration of the information processing apparatus 20 according to the embodiment.

As illustrated in FIG. 12, the information processing apparatus 20 includes a CPU (Central Processing Unit) 901, a ROM (Read Only Memory) 902, a RAM (Random Access Memory) 903, a host bus 905, a bridge 907, an external bus 906, an interface unit 908, an input device 911, an output device 912, a storage device 913, a drive 914, a connection port 915, and a communication device 916. The information processing apparatus 20 may include, instead of or together with the CPU 901, a processing circuit, such as an electric circuit, a DSP or an ASIC.

The CPU 901 functions as an arithmetic logic unit and a control device and controls overall internal operations of the information processing apparatus 20 according to various programs. The CPU 901 may be a microprocessor. The ROM 902 stores programs and operational parameters that are used by the CPU 901. The RAM 903 temporarily stores the programs that are used for execution by the CPU 901 and the parameters that vary as appropriate during the execution. The CPU 901 may, for example, fulfill the functions of the acquisition unit 201, the analyzer 203, the data compression processor 207, the learning unit 211 and the determination unit 215.

The CPU 901, the ROM 902 and the RAM 903 are mutually connected via the host bus 905 including a CPU bus. The host bus 905 is connected to the external bus 906, such as a PCI (Peripheral Component Interconnect/Interface) bus, via the bridge 907. The host bus 905, the bridge 907, and the external bus 906 may be not necessarily configured separately and a single bus may fulfill these functions.

The input device 911 is, for example, a device via which the user inputs information, such as a mouse, a keyboard, a touch panel, a button, a microphone, a switch or a lever. Alternatively, the input device 911 may be a remote control device using infrared rays and other radio waves or an external connection device, such as a mobile phone, a PDA or the like, corresponding to operations of the information processing apparatus 20. The input device 911 may, for example, include an input control circuit that generates an input signal based on information that is input by the user with the above-described input unit.

The output device 912 is a device capable of notifying the user of information visually or auditorily. The output device 912 may be, for example, a display device, such as a CRT (Cathode Ray Tube) display device, a liquid crystal display device, a plasma display device, an EL (ElectroLuminescene) display device, a laser projector, an LED (Light Emitting Diode) projector or a lamp, or may be an audio output device, such as a speaker or a headphone.

The output device 912 may, for example, output the result obtained by various types of processing performed by the information processing apparatus 20. Specifically, the output device 912 may visually display the result obtained by the information processing apparatus 20 by performing the various types of processing in various forms, such as texts, an image, a table or a graph. The output device 912 may convert an audio signal, such as sound data or acoustic data, into an analog signal and output the analog signal auditorily. The input device 911 and the output device 912 may, for example, fulfill functions of the interface unit 209.

The storage device 913 is a device for storing data that is formed as an exemplary storage of the information processing apparatus 20. The storage device 913 may be, for example, enabled using a magnetic storage device, such as a HDD (Hard Disk Drive), a semiconductor storage device, an optical storage device, or a magnetooptical storage device. For example, the storage device 913 may include a storage medium, a recording device that records data in the storage medium, a read device that reads data from the storage medium, and a deletion device that deletes data that is recorded in the storage medium. The storage device 913 may store programs that are executed by the CPU 901, various types of data, and various types of data that are acquired from the outside. The storage device 913 may, for example, fulfill the functions of the reference spectrum storage 205 and the learning model storage 213.

The drive 914 is a storage medium reader-writer and is incorporated in or externally attached to the information processing apparatus 20. The drive 914 reads the information that is recorded in a mounted removable storage medium, such as a magnetic disk, an optical disk, a magnetooptical disk or a semiconductor memory, and outputs the information to the RAM 903. The drive 914 is able to write information in the removable storage medium.

The connection port 915 is an interface that is connected to an external device. The connection port 915 is a connection port enabling data transmission to and from external devices, and the connection port 915 may be, for example, a USB (Universal Serial Bus).

The communication device 916 may be, for example, an interface that is formed of a communication device for connection with a network 40, etc. The communication device 916 may be, for example, a communication card for wired or wireless LAN (Local Area Network), LTE (Long Term Evolution), Bluetooth (trademark), or WUSB (Wireless USB). The communication device 916 may be a router for optical communication, a router for ADSL (Asymmetric Digital Subscriber Line), or various communications. The communication device 916 is, for example, able to transmit and receive signals, etc., to and from the Internet or another communication device according to a given protocol, such as TCP/IP.

The network 40 is a wired or wireless transmission path for information. For example, the network 40 may include a public network, such as the Internet, a telephone network or a satellite network, and various types of LAN (Local Area Network) or WAN (Wide Area Network) including Ethernet (trademark). The network 40 may include a dedicated line network, such as IP-VPN (Internet Protocol-Virtual Private Network).

It is also possible create a computer program for hardware, such as the CPU, ROM and RAM that are incorporated in the information processing apparatus 20, to fulfill functions equivalent to each configuration of the information processing apparatus 20 according to the above-described embodiment. Furthermore, a storage medium that stores the computer program can be provided.

The above-described embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

With reference to the accompanying drawings, preferred embodiments of the disclosure have been described in detail; however, the technical scope of the disclosure is not limited to the examples. It is obvious that those having general knowledge of the technical field of the disclosure can reach various exemplary changes and corrections within the scope of technical idea that is described in claims and it is naturally understood that the various changes and corrections belong to the technical scope of the disclosure.

The effects described herein are explanatory and exemplary only and are not definitive. In other words, the technique according to the disclosure may fulfill the above-described effects and fulfill, instead of the above-described effects, other effects obvious to those skilled in the art from the description herein.

The following configurations are within the technical scope of the present application.

(1)

An information processing system including:
at least one hardware processor; and
at least one non-transitory computer-readable storage medium storing processor-executable instructions that, when executed by the at least one hardware processor, cause the at least one hardware processor to perform:
applying a data compression process to data indicating light emitted from biological particles;
outputting, based on a result of the data compression process, one or more groups of the biological particles to sort into additional groups of the biological particles; and
using at least some of the data corresponding to the one or more groups of the biological particles in training at least one statistical model, wherein an output of the at least one statistical model specifies an indication to sort one or more of the biological particles.
   (2)

The information processing system of (1), wherein applying the data compression process to the data indicating light emitted from biological particles further includes performing clustering of the data to classify one or more of the biological particles into a plurality of groups.

(3)

The information processing system of (1), wherein applying the data compression process to the data indicating light emitted from biological particles further includes reducing a number of dimensions of the data.

(4)

The information processing system of (1), wherein the at least one hardware processor is further configured to perform:
receiving an input selecting a first group from among the one or more groups of the biological particles, and
wherein using at least some of the data further includes using data corresponding to the first group.
   (5)

The information processing system of (4), wherein receiving the input further includes receiving user input from a user interface indicating selection of the first group.

(6)

The information processing system of (1), wherein the at least one hardware processor is further configured to perform:
receiving, from a user interface, input specifying a range for at least one group of the one or more groups of the biological particles, and
wherein using at least some of the data further includes using data corresponding to the range for the at least one group.
   (7)

The information processing system of (1), wherein the data indicating light emitted from biological particles includes information received by a flow cytometer.

(8)

The information processing system of (1), wherein the data indicating light emitted from biological particles includes information identifying a spectrum of light for each of one or more biological particles.

(9)

The information processing system of (1), wherein the biological particles include at least one biological particle chosen from a cell, a microorganism, a virus, a fungus, an organelle, and a biological polymer.

(10)

The information processing system of (1), wherein one or more of the biological particles is labeled with a fluorescent dye.

(11)

The information processing system of (1), wherein the at least one statistical model includes a classifier chosen from a random forest classifier and a support vector machine classifier.

(12)

The information processing system of (1), wherein the output of the at least one statistical model identifies at least some of the data indicating light emitted from biological particles as being within a range.

(13)

An information processing method, including:
applying a data compression process to data indicating light emitted from biological particles;
outputting, based on a result of the data compression process, one or more groups of the biological particles to sort into additional groups of the biological particles; and
using at least some of the data corresponding to the one or more groups of the biological particles in training at least one statistical model, wherein an output of the at least one statistical model specifies an indication to sort one or more of the biological particles.
   (14)

At least one non-transitory computer-readable storage medium storing processor-executable instructions that, when executed by at least one hardware processor, cause the at least one hardware processor to perform:
applying a data compression process to data indicating light emitted from biological particles;
outputting, based on a result of the data compression process, one or more groups of the biological particles to sort into additional groups of the biological particles; and
using at least some of the data corresponding to the one or more groups of the biological particles in training at least one statistical model, wherein an output of the at least one statistical model specifies an indication to sort one or more of the biological particles.
   (15)

A sorting system including:
a photodetector array configured to receive light emitted from one or more biological particles;
at least one hardware processor; and
at least one non-transitory computer-readable storage medium storing processor-executable instructions that, when executed by the at least one hardware processor, cause the at least one hardware processor to perform:
   obtaining data indicating the light received by the photodetector array;
   using the data and at least one statistical model to generate an output specifying an indication to sort one or more of the biological particles, wherein the at least one statistical model was trained using training data corresponding to one or more groups of biological particles determined based on a compressed format of the training data; and
   controlling a sorting apparatus based, at least in part, on the output to sort at least some of the biological particles.
      (16)

The sorting system of (15), wherein the sorting apparatus is a flow cytometer configured to perform sorting of the biological particles based, at least in part, on the output.

(17)

The sorting system of (15), wherein the data indicating the light received by the photodetector array includes information identifying a spectrum of light for each of one or more biological particles.

(18)

The sorting system of (15), wherein the compressed format of the training data includes a plurality of groups of the biological particles generated by performing a clustering process on the training data.

(19)

The sorting system of (15), wherein the compressed format of the training data includes data having fewer dimensions than the training data.

(20)

The sorting system of (15), wherein controlling the sorting apparatus based, at least in part, on the output further includes separating a first biological particle into a first group of biological particles.

(21)

The sorting system of (20), wherein controlling the sorting apparatus based, at least in part, on the output further includes separating a second biological particle into a second group of biological particles.

(22)

The sorting system of (15), wherein the at least one processor is further configured to perform:
applying a data compression process to the data indicating light received by the photodetector array;
outputting, based on a result of the data compression process, the one or more groups of the biological particles; and
using at least some of the data corresponding to the one or more groups of the biological particles as the training data to train the at least one statistical model.
   (23)

The sorting system of (15), wherein the sorting system further includes the sorting apparatus.

(24)

An information processing method, including:
obtaining data indicating light emitted from biological particles and received by a photodetector array;
using the data and at least one statistical model to generate an output specifying an indication to sort one or more of the biological particles, wherein the at least one statistical model was trained using training data corresponding to one or more groups of biological particles determined based on a compressed format of the training data; and
controlling a sorting apparatus based, at least in part, on the output to sort at least some of the biological particles.
   (25)

The information processing method of (24), wherein the data includes information identifying a spectrum of light for each of one or more biological particles.

(26)

The information processing method of (24), wherein the biological particles include at least one biological particle chosen from a cell, a microorganism, a virus, a fungus, an organelle, and a biological polymer.

(27)

The information processing method of (24), wherein one or more of the biological particles is labeled with a fluorescent dye.

(28)

The information processing method of (24), further including:
applying a data compression process to the data;
outputting, based on a result of the data compression process, the one or more groups of the biological particles; and
using at least some of the data corresponding to the one or more groups of the biological particles as the training data to train the at least one statistical model.
   (29)

The information processing method of (28), further including:
receiving an input selecting a first group from among the one or more groups of the biological particles, and
wherein using at least some of the data further includes using data corresponding to the first group.
   (30)

At least one non-transitory computer-readable storage medium storing processor-executable instructions that, when executed by at least one hardware processor, cause the at least one hardware processor to perform:
obtaining data indicating light emitted from biological particles and received by a photodetector array;
using the data and at least one statistical model to generate an output specifying an indication to sort one or more of the biological particles, wherein the at least one statistical model was trained using training data corresponding to one or more groups of biological particles determined based on a compressed format of the training data; and
controlling a sorting apparatus based, at least in part, on the output to sort at least some of the biological particles.
   (31)

The at least one non-transitory computer-readable storage medium of (30), wherein the at least one statistical model includes a classifier chosen from a random forest classifier and a support vector machine classifier.

(32)

The at least one non-transitory computer-readable storage medium of (30), wherein the compressed format of the training data includes a plurality of groups of the biological particles generated by performing a clustering process on the training data.

(33)

The at least one non-transitory computer-readable storage medium of (30), wherein the compressed format of the training data includes data having fewer dimensions than the training data.

The following configurations are also within the technical scope of the present application.

(1)

A separation apparatus comprising:
a learning unit configured to perform data compression on optical information from biological particles, perform machine learning using the optical information before the performing the data compression on the biological particles to be separated that are specified based on information obtained by performing the data compression, and thus construct a learning model to determine optical information that is emitted from the biological particles to be separated; and
an output unit configured to output the learning model.
   (2)

The separation apparatus according to (1), wherein the data compression is clustering.

(3)

The separation apparatus according to (1), wherein the data compression is dimensional compression.

(4)

The separation apparatus according to (3), wherein the dimensional compression compresses dimensions of the optical information from the biological particles into three dimensions or less.

(5)

The separation apparatus according to any one of (1) to (4), wherein the optical information is information obtained by performing fluorescent separation on fluorescence from the biological particles to derive a level of expression of fluorescent dye of each color.

(6)

The separation apparatus according to (5), wherein the fluorescent separation is performed by a least-squares method.

(7)

The separation apparatus according to any one of (1) to (6), wherein the machine learning is supervised learning.

(8)

The separation apparatus according to (7), wherein an algorithm of the machine learning is random forests.

(9)

The separation apparatus according to any one of (1) to (8), wherein the biological particles are divided into multiple groups and then separation is performed.

(10)

The separation apparatus according to any one of (1) to (9), further comprising an interface unit configured to represent the information after the performing the data compression to a user.

(11)

The separation apparatus according to (10), wherein the interface unit is configured to map the information after the performing the data compression to an area of three dimensions or less and thus represent the information to the user.

(12)

The separation apparatus according to (10) or (11), wherein the interface unit is configured to perform the same processing on a group of the biological particles that are used for the machine learning and the separated group of biological particles and then give a visual representation to the user.

(13)

The separation apparatus according to any one of (1) to (12), wherein the learning unit is configured to, when the number of the biological particles used for the machine learning or a ratio of the number of the biological particles used for the machine learning to the whole exceeds a threshold, make a notification indicating completion of the machine learning.

(14)

The separation apparatus according to any one of (1) to (12), wherein the learning unit is configured to, when a rate of correct answers of the learning model exceeds a threshold, make a notification indicating completion of the machine learning.

(15)

The separation apparatus according to any one of (1) to (14), wherein the biological particles are cells.

(16)

A separation system comprising:
a separation apparatus configured to apply rays of light to biological particles and, based on fluorescence from the biological particles, separate the biological particles,
wherein the separation apparatus is configured to separate the biological particles that are determined by a computer as biological particles to be separated, the computer being caused to read a program that causes the computer to function as
a learning unit configured to perform data compression on optical information from biological particles, perform machine learning using the optical information before the performing the data compression on the biological particles to be separated that are specified based on information obtained by performing the data compression, and thus construct a learning model to determine optical information that is emitted from the biological particles to be separated; and
an output unit configured to output the learning model.
   (17)

A program that is read by a computer and thus causes the computer to function as a learning unit configured to perform data compression on optical information from biological particles, perform machine learning using the optical information before the performing the data compression on the biological particles to be separated that are specified based on information obtained by performing the data compression, and thus construct a learning model to determine optical information that is emitted from the biological particles to be separated.

### REFERENCE SIGNS LIST

1, 1A Sorting system
10 Sorting apparatus
11 Light source
13 Flow path
15A, 15B, 15C Dichroic mirror
16 Prism
17A, 17B, 17C Photodetector
18 Photodetector array
20, 20A Information processing apparatus
30A Information Processing Server
40 Network
201 Acquisition unit
203 Analyzer
205 Reference spectrum storage
207 Data compression processor
209 Interface unit
211 Learning unit
213 Learning model storage
215 Determination unit

1. A sorting system comprising:
at least one hardware processor; and
at least one non-transitory computer-readable storage medium storing processor-executable instructions that, when executed by the at least one hardware processor, cause the at least one hardware processor to perform:
applying a nonlinear process to optical data indicative of light emitted from biological particles to generate compressed data;
generating a learning model from the optical data and information regarding sorting of the biological particles specified based on the compressed data; and
configuring the sorting system to apply the learning model to subsequent optical data indicative of light emitted from subsequent biological particles and sort the subsequent biological particles based on result of applying the learning model.

2. The sorting system according to claim 1, wherein the nonlinear process is dimensional compression.

3. The sorting system according to claim 2, wherein the dimensional compression compresses dimensions of the optical data into three dimensions or less.

4. The sorting system according to claim 1, wherein the optical data is information obtained by performing fluorescent separation on the light emitted from the biological particles to obtain a level of expression of fluorescent dye of each color of a plurality of colors.

5. The sorting system according to claim 4, wherein the fluorescent separation is performed by a least-squares method.

6. The sorting system according to claim 1, wherein generating a learning model from the optical data and the information regarding sorting of the biological particles further comprises generating training data from the optical data and the information regarding sorting of the biological particles and training the model using the training data.

7. The sorting system according to claim 6, wherein training the model comprises performing supervised learning using the training data.

8. The sorting system according to claim 1, wherein the information regarding sorting of the biological particles is information indicating whether to sort the biological particles.

9. The sorting system according to claim 1, wherein the information regarding sorting of the biological particles is information indicating to which collection unit the biological particles are to be sorted.

10. The sorting system according to claim 1, wherein generating a learning model from the optical data and information regarding sorting of the biological particles specified based on the compressed data further comprises determining whether a rate of correct answers of the generated learning model exceeds a threshold value.

11. The sorting system according to claim 10, wherein generating a learning model from the optical data and information regarding sorting of the biological particles specified based on the compressed data further comprises dividing the optical data into first and second portions, generating the learning model using the first portion of the optical data and calculating the rate of correct answers by applying the learning model to the second portion of the optical data.

12. The sorting system according to claim 6, wherein generating the learning model from the optical data and information regarding sorting of the biological particles specified based on the compressed data further comprises generating a notification indicating completion of the machine learning when a rate of correct answers of the learning model exceeds a threshold.

13. The sorting system according to claim 1, wherein the at least one hardware processor is further configured to perform:
outputting the compressed data.

14. The sorting system according to claim 13, wherein outputting the compressed data further comprising mapping the compressed data to an area of three dimensions or less.

15. The sorting system according to claim 1, wherein the at least one hardware processor is further configured to perform:
applying the non-linear process to the optical data indicative of light emitted from the biological particles used for generating the learning model and the subsequent optical data indicative of light emitted from the subsequent biological particles to generate a second set of compressed data, and
outputting the second set of compressed data.

16. The sorting system according to claim 1, wherein the biological particles are cells.

17. The sorting system according to claim 1, further comprising:
a light source configured to irradiate laser light to the biological particles; and
a photodetector configured to obtain the light emitted from the biological particles, the light being emitted in response to irradiating the biological particles with the laser light.

18. The sorting system according to claim 17, wherein the photodetector is a photodetector array in which a plurality of photoelectric conversion elements are arranged in an array, and the photodetector array is configured to detect the light emitted from the biological particles by spectrally separating fluorescence from the biological particles.

19. A method comprising:
applying a nonlinear process to optical data indicative of light emitted from biological particles to generate compressed data;
generating a learning model from the optical data and information regarding sorting of the biological particles specified based on the compressed data; and
configuring a sorting system to apply the learning model to subsequent optical data indicative of light emitted from subsequent biological particles and sort the subsequent biological particles based on result of applying the learning model.

20. At least one non-transitory computer-readable storage medium storing processor-executable instructions that, when executed by the at least one hardware processor, cause the at least one hardware processor to perform:
applying a nonlinear process to optical data indicative of light emitted from biological particles to generate compressed data;
generating a learning model from the optical data and information regarding sorting of the biological particles specified based on the compressed data; and
configuring a sorting system to apply the learning model to subsequent optical data indicative of light emitted from subsequent biological particles and sort the subsequent biological particles based on result of applying the learning model.
